(19) United States
(12) Patent Application Publication
(10) Pub. No.: US 2026/0290556 A1
(43) Pub. Date: Sep. 24, 2026

(54) Method and Systems for Filtering, Sorting, and Displaying Images Using Computer Aided Detection

(71) Applicant: FUJIFILM Healthcare Americas Corporation, Lexington, MA (US)

(72) Inventor: Azuma Takahashi, Cary, NC (US)

(73) Assignee: FUJIFILM Healthcare Americas Corporation, Lexington, MA (US)

(21) Appl. No.: 19/566,256

(22) Filed: Mar. 13, 2026

Related U.S. Application Data
(63) Continuation of application No. 18/514,163, filed on Nov. 20, 2023, now abandoned.

(51) Int. Cl. G16H 30/20; G06T 7/00; G16H 30/40

(52) U.S. Cl. CPC G16H 30/20; G06T 7/0012; G16H 30/40; G06T 2207/20024; G06T 2207/30096

(57) ABSTRACT

Methods and system for filtering, sorting, and displaying images are provided. The method can include receiving a request to view two or more images associated with a patient and receiving a request to perform Computer Aided Detection and Diagnosis (CAD) on the two or more images. The method can include performing CAD on the two or more images. Performing CAD can include identifying at least one characteristic of each of the two or more images, respectively. The method can include sorting the two or more images to generate an image order, the image order determined based at least in part on the at least one characteristic of each of the two or more images. The two or more images can be transferred from the one or more computing devices to the client device, and the two or more images can be displayed in the image order.

### BACKGROUND

### 1. Field of Disclosed Subject Matter

The disclosed subject matter is directed to systems and methods for filtering, sorting, and displaying images based on Computer Aided Detection and Diagnosis (“CAD”), for example, filtering, sorting, and displaying Digital Imaging and Communications in Medicine (“DICOM”) images.

### 2. Description of Related Art

In medical imaging, Picture Archiving and Communication Systems (“PACS”) are a combination of computers and networks dedicated to the storage, retrieval, presentation, and distribution of images. While medical information can be stored in a variety of formats, a common format of image storage is DICOM. DICOM is a standard in which, among other things, medical images and associated meta-data can be communicated from imaging modalities (e.g., x-ray (or x-rays' digital counterparts: computed radiography (“CR”) and digital radiography (“DR”)), computed tomography (“CT”), and magnetic resonance imaging (“MRI”) apparatuses) to remote storage and/or client devices for viewing and/or other use.

When viewing medical images, CAD can be utilized by practitioners to help identify certain features, such as diseases within the images. For example, CADt can aid in the prioritization and triage of medical images. CADe can detect diseases, such as lung nodules and bone fractures. CADx can identify and characterize disease features, such as whether the disease is benign or malignant. A list function can provide results to a user in a list. However, during use, CAD programs can provide inefficient results. For example, CADe can detect too many disease candidates (e.g., more than 10) and lists can include too many results. When several CAD programs are executed, results can be even more inefficient.

Accordingly, there is a need to improve efficiency presentation of images analyzed by CAD programs, for example, by sorting and/or filtering results. Such sorting and/or filtering can be based on disease size, features, artificial intelligence (AI) confidence level (e.g., based on image background), user preference, and diagnostic guidelines.

### SUMMARY

The purposes and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as the appended figures.

To achieve these and other advantages and in accordance with the purpose of the disclosed subject matter, as embodied and broadly described the disclosed subject matter is directed to systems and methods for filtering, sorting, and displaying images using computer aided detection. The method includes receiving, by one or more computing devices and from a client device of a first user, a request to view two or more images associated with a patient. The method includes receiving, by the one or more computing devices and from the client device of the first user, a request to perform Computer Aided Detection and Diagnosis (CAD) on the two or more images. The method further includes performing, by the one or more computing devices, CAD on the two or more images, wherein performing CAD includes identifying at least one characteristic of each of the two or more images, respectively. The method include sorting, by the one or more computing devices, the two or more images to generate an image order, the image order determined based at least in part on the at least one characteristic of each of the two or more images. The method includes transferring the two or more images from the one or more computing devices to the client device; and displaying, by the client user device, at least one of the two or more images, wherein the displayed images are displayed in the image order.

In accordance with the disclosed subject matter, the two or more images can each include Digital Imaging and Communications in Medicine (“DICOM”) images. The one or more computing devices can include a picture archiving and communication system (PACS). The respective characteristic of each of the two or more images can be a disease. For example, the disease can be a plurality of lesions.

In accordance with the disclosed subject matter, the method can include classifying each respective disease as benign or malignant. Sorting can be based at least in part on the classifications. The respective characteristic of each of the two or more images can be an anatomy type. The image order can be based on a plurality of characteristics of each of the two or more images.

The method can include filtering, by the one or more computing devices, the two or more images. The filtering can be determined based at least in part on the at least one characteristic of each of the two or more images. The displayed images can be displayed based on the filtering.

In accordance with the disclosed subject matter, the method can include grouping, by the one or more computing devices, the two or more image. The grouping can be determined based at least in part on the at least one characteristic of each of the two or more images. The displayed images can be displayed based on the grouping. The method can include identifying at least one image associated with the characteristic of at least one of the two or more images. Sorting can be based on a preference of the user.

### Drawings

FIG. 1 shows a hierarchy of medical image records that can be filtered and displayed in accordance with the disclosed subject matter.

FIG. 2 shows the architecture of a system for filtering, sorting, and displaying images, in accordance with the disclosed subject matter.

FIG. 3 shows a graphical user interface in accordance with the disclosed subject matter.

FIG. 4 shows a flow chart of a method in accordance with the disclosed subject.

### DETAILED DESCRIPTION

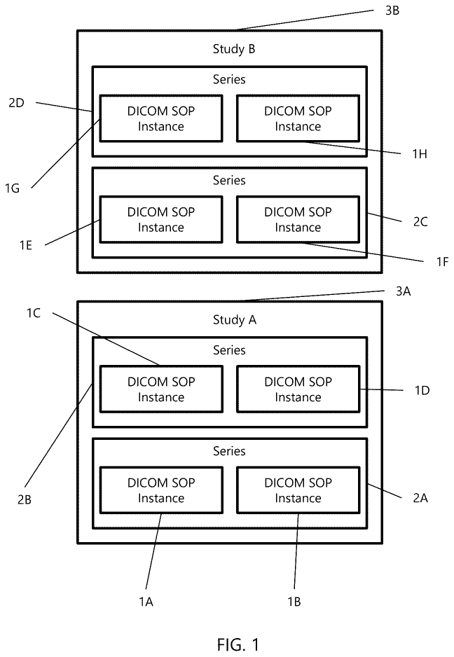

Reference will now be made in detail to various exemplary embodiments of the disclosed subject matter, exemplary embodiments of which are illustrated in the accompanying drawings. For purpose of illustration and not limitation, the systems and method are described herein with respect to filtering, sorting, and displaying images, and particularly, digital medical images (also referred to as “medical images”), such as DICOM images. However, the methods and systems described herein can be used for filtering, sorting, and displaying any digital image. As used in the description and the appended claims, the singular forms, such as “a,” “an,” “the,” and singular nouns, are intended to include the plural forms as well, unless the context clearly indicates otherwise. Accordingly, as used herein, the term medical image can refer to one medical image, or a plurality of medical images. For example, and with reference to FIG. 1 for purpose of illustration and not limitation, as referred to herein a medical image record can include a single DICOM Service-Object Pair (“SOP”) Instance (also referred to as “DICOM Instance” “DICOM image” and “image”) 1 (e.g., 1A-1H), one or more DICOM SOP Instances 1 in one or more Series 2 (e.g., 2A-D), one or more Series 2 inside one or more Studies 3 (e.g., 3A, 3B), and one or more Studies 3. A DICOM SOP Instance 1 (e.g., 1A-1H) can be mammogram.

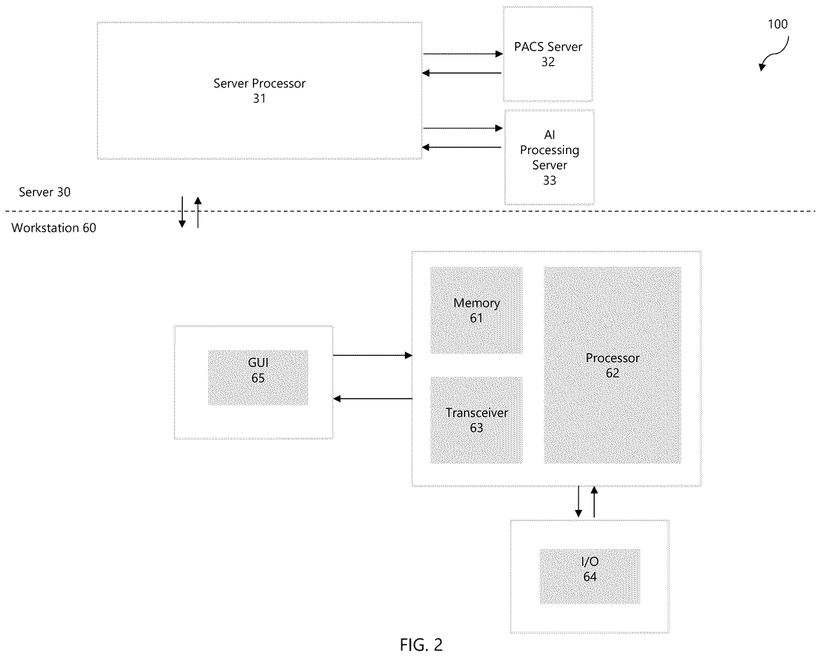

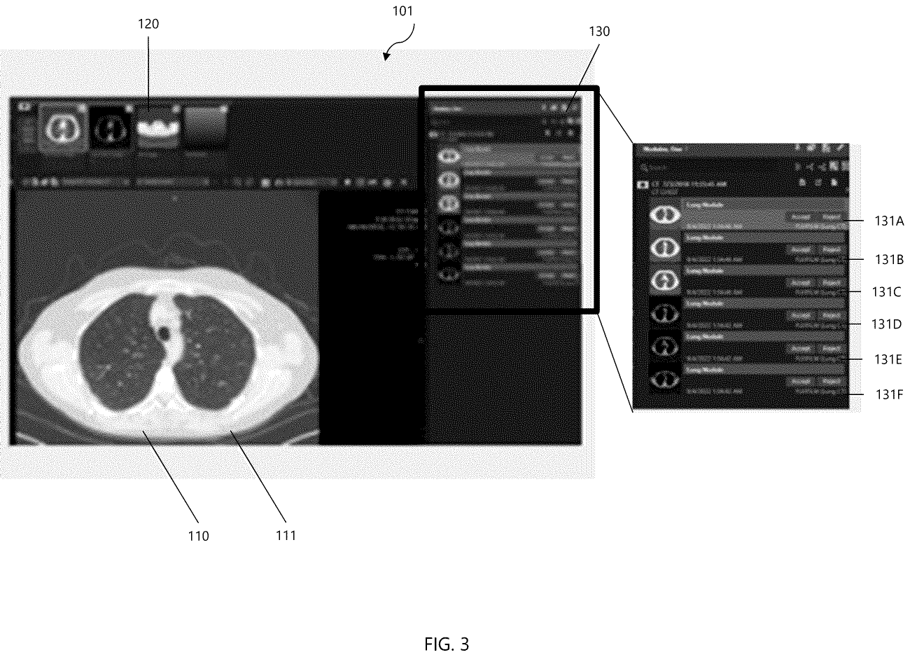

Referring to FIGS. 2-3 for purpose of illustration and not limitation, the disclosed system 100 can be configured to filter and/or sort images. For example, system 100 can be configured for filtering and/or sorting medical images that have been analyzed by CAD programs, such as DICOM images (e.g., 1A-1H). The system 100 can include one or more computing devices defining a server 30 and user workstation 60. The user workstation 60 can be coupled to the server 30 by a network. The network, for example, can be a Local Area Network (“LAN”), a Wireless LAN (“WLAN”), a virtual private network (“VPN”), or any other network that allows for any radio frequency or wireless type connection. For example, other radio frequency or wireless connections can include, but are not limited to, one or more network access technologies, such as Global System for Mobile communication (“GSM”), Universal Mobile Telecommunications System (“UMTS”), General Packet Radio Services (“GPRS”), Enhanced Data GSM Environment (“EDGE”), Third Generation Partnership Project (“3GPP”) Technology, including Long Term Evolution (“LTE”), LTE-Advanced, 3G technology, Internet of Things (“IOT”), fifth generation (“5G”), or new radio (“NR”) technology. Other examples can include Wideband Code Division Multiple Access (“WCDMA”), Bluetooth, IEEE 802.11b/g/n, or any other 802.11 protocol, or any other wired or wireless connection.

Workstation 60 can take the form of any known client device. For example, workstation 60 can be a computer, such as a laptop or desktop computer, a personal data or digital assistant (“PDA”), or any other user equipment or tablet, such as a mobile device or mobile portable media player. Server 30 can be a service point which provides processing, database, and communication facilities. For example, the server 30 can include dedicated rack-mounted servers, desktop computers, laptop computers, set top boxes, integrated devices combining various features, such as two or more features of the foregoing devices, or the like. Server 30 can vary widely in configuration or capabilities, but can include one or more server processors 31, PACS server 32, AI processing server 33, memory, and/or transceivers. Although illustrated as separate elements, one or more of the server processors 31, PACS server 32, AI processing server 33 can be combined. Server 30 can also include one or more mass storage devices, one or more power supplies, one or more wired or wireless network interfaces, one or more input/output interfaces, and/or one or more operating systems.

A user can be any person authorized to access workstation 60 and/or server 30, including a health professional, medical technician, researcher, or patient. In some embodiments a user authorized to use the workstation 60 and/or communicate with the server 30 can have a username and/or password that can be used to login or access workstation 60 and/or server 30.

Workstation 60 can include GUI 65, memory 61, processor 62, transceiver 63, and input/output 64. Medical image records (such as mammograms) received by workstation 60 can be processed using one or more processors 62. Processor 62 can be any hardware or software used to execute computer program instructions. These computer program instructions can be provided to a processor of a general purpose computer to alter its function to a special purpose, a special purpose computer, application-specific integrated circuit (“ASIC”), or other programmable digital data processing apparatus, such that the instructions, which execute via the processor of the workstation 60 or other programmable data processing apparatus, implement the functions/acts specified in the block diagrams or operational block or blocks, thereby transforming their functionality in accordance with embodiments herein. The processor 62 can be a portable embedded micro-controller or micro-computer. For example, processor 62 can be embodied by any computational or data processing device, such as a central processing unit (“CPU”), digital signal processor (“DSP”), ASIC, programmable logic devices (“PLDs”), field programmable gate arrays (“FPGAs”), digitally enhanced circuits, or comparable device or a combination thereof. The processor 62 can be implemented as a single controller, or a plurality of controllers or processors. The input/output 64 can be any suitable input/output, for example, a mouse or keyboard. In accordance with the disclosed subject matter, input/output 64 can be integrated with GUI 65 in the form of a touchscreen. Input/output 64 can be implemented as a single input/output 64 or a plurality of input/outputs 64.

Workstation 60 can send and receive medical image records (such as CT scans) from server 30 using transceiver 63. Transceiver 63 can, independently, be a transmitter, a receiver, or both a transmitter and a receiver, or a unit or device that can be configured both for transmission and reception. In other words, transceiver 63 can include any hardware or software that allows workstation 60 to communicate with server 30. Transceiver 63 can be either a wired or a wireless transceiver. When wireless, the transceiver 63 can be implemented as a remote radio head which is not located in the device itself, but in a mast. While FIG. 2 only illustrates a single transceiver 63, workstation 60 can include one or more transceivers 63. Memory 61 can be a non-volatile storage medium or any other suitable storage device, such as a non-transitory computer-readable medium or storage medium. For example, memory 61 can be a random-access memory (“RAM”), read-only memory (“ROM”), hard disk drive (“HDD”), erasable programmable read-only memory (“EPROM”), electrically erasable programmable read-only memory (“EEPROM”), flash memory or other solid-state memory technology. Memory 61 can also be a compact disc read-only optical memory (“CD-ROM”), digital versatile disc (“DVD”), any other optical storage, magnetic cassettes, magnetic tape, magnetic disk storage or other magnetic storage devices, or any other physical or material medium which can be used to tangibly store the desired information or data or instructions and which can be accessed by a computer or processor. Memory 61 can be either removable or non-removable.

FIG. 3 shows, for purpose of illustration and not limitation, GUI 65. GUI 65 can be controlled by one or more computing devices which can be on the workstation 60, server 30, or both. GUI 65 can include display area 110 (showing medical image 111, which is a CT scan), thumbnail display area 120, and result list 130. Result list can include a plurality of study icons 131 (e.g., 131A-131F).

In operation, system 100 can receive a request form a user (for example, a doctor, nurse, or other medical practitioner) to view two or more medical images (e.g., 111) and to perform CAD one the two or more medical images (e.g., 111). The user can provide the request at workstation 60 via input/output 64. The request can be transferred from the workstation 60 to server 30. At server 30, the appropriate medical images (e.g., 111) can be identified (e.g., by one or more of service processor 31, PACS server 32, and/or AI processing server 33) and the images can be processed by CAD (e.g., by one or more of service processor 31, PACS server 32, and/or AI processing server 33). The CAD processing can analyze image background, whether the image is follow-up, initial screening, or incidental image, what the study objection was in the hospital information system (HIS), and reconstruction condition. Additionally or alternatively, the system can analyze the image property using DICOM tag information (for example, image reconstruction condition, slice thickness, and study description, or other suitable DICOM tag information). The CAD results can be used to filter and/or sort the medical images (e.g., 111).

The CAD operation can be one or more types of CAD operations, including CADe (for disease detection), CADx (for disease identification and characterization), and CADt (for prioritization and triage of medical images). For example, CAD can produce a collection of images with certain diseases identified (e.g., pulmonary nodules or rib fractures).

The results can be processed by one or more of service processor 31, PACS server 32, and/or AI processing server 33 to filter and/or sort the results. Filtering and/or sorting can be based on the image background and/or user preference preset setting. In accordance with the disclosed subject matter, filtering and/or sorting can be based on an algorithm. The algorithm can be partially or completely hard coded. As another example, AI technology can be used to learn how to use AI for diagnosis and screening, and therefore filtering and/or sorting. For example, a doctor can change the filtering and/or sorting condition for a specific image (or many images). The system can update the changing log. Based on the changing log, AI technology can learn how to decide the appropriate filter and/or sorting condition for particular images. The images and filtering/sorting information can be transferred from the server 30 to workstation 60. The results can be displayed in result list 130 as study icons 131 (e.g., 131A-131F), which can be selected by the user to display the respective image (e.g., 111) in display area 110.

Sorting and filtering can be based on a variety of criteria. For example, in the United States, the standard for detection of pulmonary nodules is 4 mm or more for screening and 6 mm or more for incidentally discovered nodules (according to the Fleishner Society guidelines). As such, images can be sorted based on nodule size and can be filtered based on the purpose of the imaging (e.g., filtering out nodules less than 4 mm for screening and filtering out nodules less than 6 mm for incidental discovery). As another example, images can be filtered/sorted based on the size of the detected disease (e.g., average diameter, length volume, area), size change as compared to past examinations, lesion type (e.g., benign or malignant), guidelines, or location of the image. Sorting by lesion type can be particularly useful if CADx can be used for disease classification. Multiple levels can also be used. For example, images can first be sorted by benign v. malignant (e.g., with malignant cases moved above benign cases) and then by size (e.g., with larger malignant cases moved above smaller malignant cases, then larger benign cases moved above smaller benign cases). Additionally, or alternatively, sorting can be based on the seriousness of the injury. For example, in an emergency medicine scenario, a skull fracture can give priority over a rib fracture. Such a hierarchy can be modified due to complications, such as a rib fracture in combination with large pleural effusion. As another example, pulmonary nodules that are jagged (spicule) or large in size (which can indicate the nodules are malignant and have high metastasis) can be given priority over smoother and/or smaller nodules. Diseases that have rapidly increased in size compared to past examinations can also be given higher priority. In accordance with the disclosed subject matter, AI technology can be used to determine the proper priority. For example, doctors can set priority for a variety of diseases, and AI technology can learn the priority based on the settings provided by doctors.

In accordance with the disclosed subject matter, a plurality of lesions or other detected abnormalities can be grouped as a single disease and be treated as such for sorting and filtering. For example, a group of lung lesions can be treated as a single disease such as pneumonia.

Likewise diffuse axonal injury resulting from traumatic injuries, such as head trauma, compound fractures, and diseases such as hemothorax, pneumothorax, and pulmonary contusions (which are complications of rib fractures), can each be displayed as the same disease group. For example, the study icons 131 (e.g., 131A-131F) can be grouped together and only one study icon 131 (e.g., 131A) can be displayed.

When a user clicks selects a study icon 131 (e.g., 131A-131F), a corresponding image 111 can be displayed in the display area 110. Additionally, if the current image 111 is a lung condition, one or more previous images of the patient's same lung condition can be displayed in the display area 110. When displaying images 111, presets can be set based on the purpose of the image. For example, a lung condition image can be displayed to diagnose lung disease, while a bone condition image can be used to diagnose bone disease. Such changes can make reviewing the image 111 more efficient. For example, if a CT scan is a thin slice, doctors can typically detect smaller disease. Accordingly, filtering and/or sorting can be based on a slice thickness. As another example, study description can include information about the study objective, which can be used for filtering and/or sorting.

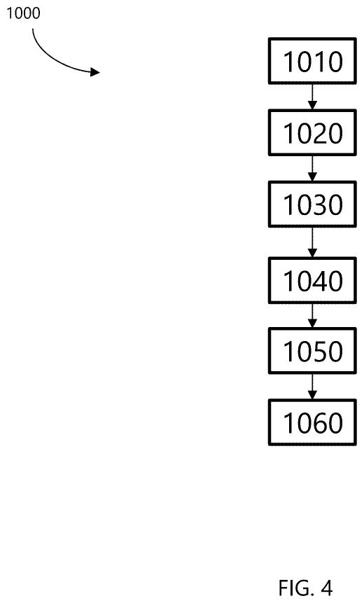

FIG. 4 illustrates an example method 1000 in accordance with the disclosed subject matter. The method can begin at step 1010, where the method includes receiving, by one or more computing devices and from a client device of a first user, a request to view two or more images associated with a patient. At step 1020, the method can include receiving, by the one or more computing devices and from the client device of the first user, a request to perform Computer Aided Detection (CAD) on the two or more images. At step 1030, the method can include performing, by the one or more computing devices, CAD on the two or more images, wherein performing CAD includes identifying at least one characteristic of each of the two or more images, respectively. At step 1040, the method can include sorting, by the one or more computing devices, the two or more images to generate an image order, the image order determined based at least in part on the at least one characteristic of each of the two or more images. At step 1050, the method can include transferring the two or more images from the one or more computing devices to the client device. At step 1060, the method can include displaying, by the client user device, at least one of the two or more images, wherein the displayed images are displayed in the image order. In accordance with the disclosed subject matter, the method can repeat one or more steps of the method of FIG. 4, where appropriate.

Although this disclosure describes and illustrates particular steps of the method of FIG. 4 as occurring in a particular order, this disclosure contemplates any suitable steps of the method of FIG. 4 occurring in any suitable order. Moreover, although this disclosure describes and illustrates an example method including the particular steps of the method of FIG. 4, this disclosure contemplates any suitable method including any suitable steps, which can include all, some, or none of the steps of the method of FIG. 4, where appropriate. Furthermore, although this disclosure describes and illustrates particular components, devices, or systems carrying out particular steps of the method of FIG. 4, this disclosure contemplates any suitable combination of any suitable components, devices, or systems carrying out any suitable steps of the method of FIG. 4.

The subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or may be included in, one or more separate physical components or media (e.g., multiple CDs, disks, or other storage devices).

The term “processor” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA or an ASIC. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC.

Processors suitable for the execution of a computer program can include, by way of example and not by way of limitation, both general and special purpose microprocessors.

Devices suitable for storing computer program instructions and data can include all forms of non-volatile memory, media and memory devices, including by way of example but not by way of limitation, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Additionally, as described above in connection with certain embodiments, certain components can communicate with certain other components, for example via a network, e.g., a local area network or the internet. To the extent not expressly stated above, the disclosed subject matter is intended to encompass both sides of each transaction, including transmitting and receiving. One of ordinary skill in the art will readily understand that with regard to the features described above, if one component transmits, sends, or otherwise makes available to another component, the other component will receive or acquire, whether expressly stated or not.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1-14. (canceled)

15. A system comprising: one or more hardware processors; a memory coupled to the processors comprising instructions executable by the processors, the processors being operable when executing the instructions to:
receive a request to perform a computer aided detection and diagnosis (“CAD”) operation on a plurality of Digital Imaging and Communications in Medicine (“DICOM”) Service-Object Pair (“SOP”) instances, each DICOM SOP instance having DICOM tag information, wherein the CAD operation includes at least one of CADe to detect a disease, CADx to characterize a disease feature, and CADt to prioritize DICOM SOP instances;
performing the CAD operation on each DICOM SOP instance of the plurality of DICOM SOP instances to identify a respective
a first image property based on the DICOM tag information, the DICOM tag information including at least one of an image reconstruction condition, a slice thickness, and a study description, and
an image characteristic;
filtering the plurality of DICOM SOP instances into a first set of DICOM SOP instances for display and a second set of DICOM SOP instance based on each respective image first image property and each respective image characteristic; and
display, on a graphical user interface, the first set of DICOM SOP instances.

16. The system of claim 15, wherein the image characteristic comprises a disease identified via disease detection.

17. The system of claim 16, wherein the disease comprises a plurality of lesions.

18. The system of claim 16, wherein filtering is based the respective disease.

19. The system of claim 16, wherein the processors are further operable when executing the instructions to classify each respective disease as benign or malignant.

20. The system of claim 19, wherein filtering is based on the classification of each respective disease.

21. The system of claim 16, wherein the characteristic further comprises a size of the respective disease.

22. The system of claim 21, wherein filtering is based on the size of each respective disease.

23. The system of claim 15, wherein filtering is based on a purpose of the DICOM SOP instances.

24. The system of claim 15, wherein the processors are further operable when executing the instructions to sort the first set of DICOM SOP instances prior to displaying the first set of DICOM SOP instances.

25. The system of claim 24, wherein sorting is based on each respective image first image property and each respective image characteristic.
